(19) United States
(12) Patent Application Publication
(10) Pub. No.: US 2025/0314531 A1
(43) Pub. Date: Oct. 9, 2025

(54) METHOD AND SYSTEM FOR MEASUREMENT OF NONTHERMAL FAR-INFRARED RADIATION

(71) Applicant: Albert Chin-Tang WEY, Westmont, IL (US)

(72) Inventor: Albert Chin-Tang WEY, Westmont, IL (US)

(21) Appl. No.: 19/245,032

(22) Filed: Jun. 20, 2025

Related U.S. Application Data
(63) Continuation-in-part of application No. 18/370,289, filed on Sep. 19, 2023.
(63) Continuation-in-part of application No. 18/199,660, filed on May 19, 2023.
Application No. 18/370,289 is a continuation-in-part of application No. 17/473,799, filed on Sep. 13, 2021.
Application No. 18/199,660 is a continuation-in-part of application No. 17/473,799, filed on Sep. 13, 2021.

(51) Int. Cl. G01J 5/00; G01J 5/08

(52) U.S. Cl. CPC G01J 5/0003; G01J 5/0859; G01J 2005/0077

(57) ABSTRACT

The present invention relates to a method and system for measuring the radiant power and emissivity of nonthermal far-infrared (FIR) radiation emitted from the surface of an FIR-photon-emitting specimen, specifically within the 3-16 μm wavelength range. This method and system include at least a test specimen, a reference material, an infrared imaging device, and a processing apparatus configured to execute a set of instructions. These instructions facilitate operations such as capturing thermal images of the test specimen and reference material, computing their grayscale values, and generating results that reveal the radiant power and emissivity of the test specimen, characteristics that would otherwise remain undetectable and imperceptible.

### CROSS REFERENCE TO RELATED APPLICATIONS

This application is a continuation-in-part application claiming the benefit of application Ser. Nos. 18/370,289 filed Sep. 19, 2023, Ser. No. 18/199,660 filed May 19, 2023, and Ser. No. 17/473,799 filed Sep. 13, 2021. All applications are incorporated by reference herein in their entirety.

### BACKGROUND

### Field of Invention

The present invention relates to a method and system for measuring the radiant power and emissivity of nonthermal far-infrared (FIR) radiation emitted from the surface of an FIR-photon-emitting specimen, specifically within the 3-16 μm wavelength range.

### Description of Prior Art

Far-infrared (FIR) therapy, utilizing nonthermal FIR-photon radiation, offers several therapeutic benefits, including promoting wound healing, alleviating pain and inflammation, enhancing immune system responses, and mitigating the side effects of cancer treatments. Some studies suggest that the absorption of nonthermal FIR photons within the 3-16 μm wavelength range induces molecular vibrations, potentially increasing the internal energy of reactants and influencing reaction rates by lowering the reaction barrier, as many bioeffects in the human body involve chemical reactions.

In therapeutic applications, identifying an effective nonthermal FIR-photon-emitting source presents a challenge, as the target wavelengths (3-16 μm) significantly overlap with the blackbody (thermal) radiation spectrum (3-25 μm) at room temperature. This overlap often leads to confusion between nonthermal FIR-photon radiation and blackbody (thermal) FIR radiation, as both occupy the same region of the infrared spectrum.

All objects on Earth emit thermal radiation to varying degrees. The thermal radiation from ordinary materials can often be approximated by blackbody radiation. A blackbody at room temperature (25° C. or 298 K) primarily emits energy in the infrared spectrum, spanning wavelengths from approximately 3 to 1000 μm, with about 90% of its energy concentrated in the 3-25 μm range and around 67% in the 3-16 μm range.

Conversely, the emission of nonthermal FIR photons requires a specific crystalline structure in which transition metal ions are positioned within coordination complexes. This arrangement induces crystal field splitting, forming FIR luminescence centers. The energy gap between the split d-orbitals of the transition metal ion corresponds to a luminescence profile spanning wavelengths from approximately 3 to 16 μm.

A common error in prior art concerns the use of the “3-16 μm” segment of the thermal radiation spectrum in applications requiring specific 3-16 μm wavelength photon luminescence. Describing thermal radiation as limited to the “3-16 μm” range is misleading. Theoretically, blackbody (thermal) radiation follows a continuous wavelength spectrum, known as the Planck spectrum, which depends solely on the temperature of the emitting body. The 3-16 μm segment is an inherent part of the broader spectrum (3-1000 μm) and cannot be considered in isolation.

That said, the absorption of thermal radiation enables radiative heat transfer to molecules in condensed matter (solids and liquids), converting it into thermal motion that excites a combination of electronic transitions, molecular vibrations, and lattice oscillations. In contrast, the absorption of nonthermal FIR photons induces specific molecular vibrations without significantly increasing the material's thermal energy.

Although thermal radiation and nonthermal FIR-photon radiation have distinct origins, they overlap within the same wavelength range (3-16 μm). Because the radiant power of nonthermal FIR photon emission is relatively low compared to that of thermal radiation, it is often masked by the thermal background. This overlap represents a major hurdle for detecting and measuring nonthermal FIR photon radiation in thermally active environments.

During the development of a nonthermal FIR-photon-emitting ceramic module for therapeutic applications, the present inventor recognized an urgent need for a reliable apparatus to measure nonthermal FIR-photon radiation. By leveraging the aforementioned spectral overlap, the present inventor devised a method to effectively detect extremely low-power, nonthermal FIR-photon radiation that would otherwise remain undetectable. This breakthrough led to the development of a novel measurement method by the present inventor (Wey, U.S. patent application Ser. No. 18/370,289).

The “Blackbody Temperature Approximation” measurement method described in the quoted invention involves positioning a test specimen adjacent to a blackbody reference source preset to a specified temperature. Electromagnetic radiation from both sources, preferably within the same image frame, is recorded using an infrared imaging device operating in the 3-16 μm wavelength range. A processing apparatus is employed to acquire, store, and analyze the image's grayscale data. Additionally, a temperature measuring apparatus is required to record the contact temperature of the test specimen for reference.

However, during the implementation of the inventive measurement method, the present inventor encountered challenges in obtaining reliable results due to fluctuations in ambient temperature. Although the innovative method proved functional, it exhibited limitations under variable conditions, prompting the present inventor to seek a more robust and dependable approach.

While conducting experiments using the newly developed measurement method, a rubber material was employed as a near-blackbody radiation reference to monitor temperature variations. In certain trials, the nonthermal FIR-photon-emitting specimen was deliberately positioned behind the rubber material. As a result, the present inventor observed an unexpected phenomenon: the rubber material exhibited transparency to nonthermal FIR-photon radiation.

This previously unrecognized phenomenon prompted the present inventor to reconsider the principle of “the equality of absorptivity and emissivity,” as described by Planck's law and Kirchhoff's law of thermal radiation, in search of an explanation.

The equation of radiative transfer describes how radiation is influenced as it propagates through a material medium. According to Kirchhoff's law, at thermodynamic equilibrium, the thermal radiation emitted by a black body has a unique, universal spectral radiance that depends solely on temperature and is accurately described by Planck's law.

The equation of radiative transfer states that, for a beam of light traveling through a small distance, energy is conserved. This change equals the energy removed from the beam plus the energy added by the medium. At equilibrium, these contributions balance, and the material's emission and absorption coefficients obey Kirchhoff's law, reflecting the equality of absorptivity and emissivity.

Moreover, the principle of detailed balance states that, under thermodynamic equilibrium, every elementary process is precisely countered by its reverse. In 1916, Albert Einstein applied this principle at the atomic level to explain how atoms emit and absorb energy through transitions between discrete energy states.

In the present inventor's case, it is posited that while the radiation field remains in equilibrium with the material medium (rubber), nonthermal photon beams may traverse it unimpeded, as they are not involved in the thermal radiation process. Under the condition of equal absorptivity and emissivity for thermal radiation within the overlapping 3-25 μm wavelength range, the rubber thereby forms a transparency window for nonthermal photons spanning 3-16 μm.

This finding simplifies the system requirements for detecting and measuring nonthermal FIR-photon radiation, thereby leading to the present invention.

The proposed method involves positioning the test specimen behind a reference material with a known thermal emissivity, preferably greater than 0.8. An infrared imaging device is then used to capture thermal images of both materials. Because rubber becomes partially transparent to nonthermal FIR photon radiation, the test specimen may appear visually distinct through the rubber in the resulting thermal images. By analyzing these images in conjunction with the known thermal properties of the reference material, the radiant power and emissivity of the test specimen can be accurately determined.

Although thermography (thermal imaging) has a well-established history, it has traditionally been associated with blackbody radiation, primarily for object detection and temperature measurement, as demonstrated in prior art (e.g., U.S. Patent and application Nos. 20230245541, 20230204429, 11604098, 20210341337, 20210123818, 10272920, 9357963, 389939, and 7795583).

Unlike prior thermographic techniques centered on blackbody (thermal) radiation, the present invention introduces a novel methodology for detecting and quantifying nonthermal FIR photon emission from a test specimen, offering capabilities not addressed in the prior art.

### Objects and Advantages

Accordingly, one object of this invention is to provide a method for detecting and measuring nonthermal FIR-photon radiation in 3-16 μm wavelength range;

Another object of this invention is to provide a simple and user-friendly system for measuring the radiant power and emissivity of a specimen that emits nonthermal FIR-photon radiation within the 3-16 μm wavelength range.

These objects are achieved through a method and system comprising, at least, a test specimen, a reference material, an infrared imaging device, and a processing apparatus configured to perform operations including acquisition, computation, and display of thermal image data; whereby the radiant power and emissivity of the test specimen's nonthermal FIR radiation can be quantified.

Other objects, features, and advantages of the present invention will hereinafter become apparent to those skilled in the art from the following description.

### SUMMARY OF THE INVENTION

In accordance with the present invention, a method and system for measuring nonthermal far-infrared radiation in 3-16 μm wavelength spectrum comprise at least a test specimen, a reference material, an infrared imaging device, and a processing apparatus configured to perform operations including acquisition, computation, and display of thermal image data; whereby the radiant power and emissivity of the test specimen's nonthermal far-infrared radiation can be quantified.

### BRIEF DESCRIPTION OF THE DRAWINGS

FIG. 1 is a schematic diagram illustrating one embodiment of the present invention, in which the test specimen is positioned directly behind the reference material.

FIG. 2 illustrates a representative thermal image obtained using the system depicted in FIG. 1.

FIG. 3 is a schematic diagram illustrating another embodiment of the present invention, wherein the test specimen is positioned within the reference material.

FIG. 4 is a schematic diagram illustrating another embodiment of the present invention, wherein multiple test specimens are positioned within the reference material to enable comparative analysis.

FIG. 5 illustrates a representative thermal image obtained using the system depicted in FIG. 4.

Reference Numerals in Drawings

| | |
|---|---|
| 11 Test Specimen A | 12 Test Specimen B |
| 13 Reference Material | 31 Infrared Imaging Device |
| 32 Processing Apparatus | 41 Image of Test Specimen A |
| 42 Image of Test Specimen B | 43 Image of Reference Material |
| 44 Image of Background Scene | |

### DETAILED DESCRIPTION OF THE INVENTION

The groundbreaking discovery by the present inventor, that most nonmetallic materials exhibit transparency to nonthermal FIR-photon radiation, serves as the foundation of this invention, enabling straightforward and accessible detection of this previously elusive emission.

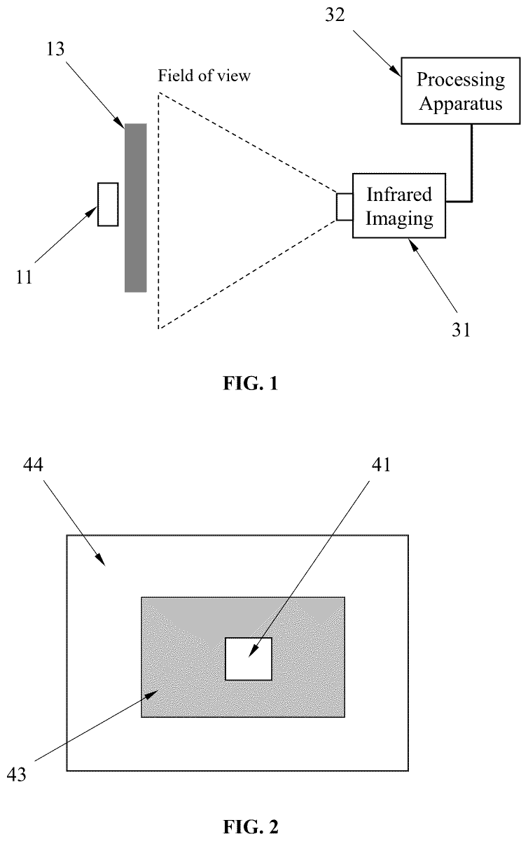

FIG. 1 shows a schematic diagram illustrating an exemplary system of the present invention for measurement of nonthermal FIR-photons radiation. The system includes at least a test specimen 11, a reference material 13, an infrared imaging device 31, and a processing apparatus 32.

Test specimen A 11 is a sample designed for evaluation of nonthermal FIR-photon emission using the method and system described herein. While all objects on Earth emit thermal radiation, only certain materials, those with a specific FIR-photon emission mechanism, can radiate nonthermal FIR photons. This mechanism typically involves a crystalline structure incorporating transition metal ions within distorted coordination complexes.

Test specimens may take various shapes, such as planar, cylindrical, or spherical, and may be of any size. However, to ensure that the thermal image accurately represents the surface-emitted radiant energy without distortion, a flat surface area of at least 5 mm×5 mm is preferred.

Reference material 13 may comprise any nonmetallic substance, including but not limited to polymers, ceramics, glass, composites, concrete, wood, or paper. Polymers, such as plastics (e.g., polyethylene, polycarbonate), rubber, or silicone, are preferred for their favorable elasticity and flexibility. However, any material with an emissivity greater than 0.8 is considered suitable.

Reference material 13 must be a nonmetal, as metals reflect and absorb thermal radiation very effectively. This is due to the high density of free electrons in metals, which respond to incident thermal radiation by oscillating and either reflecting the energy or converting it to surface heat. These interactions prevent infrared radiation from passing through. Consequently, metals exhibit very low emissivity values, such as aluminum Foil (0.04), polished brass (0.03), nickel alloy (0.06), iron (0.2), magnesium (0.1), steel (0.2), nickel (0.07), silver (0.02), Titanium (0.2), and zinc (0.05).

On the other hand, some nonmetals (such as ceramics, glass, polymers, and plastics) can transmit thermal radiation, particularly in the infrared range, depending on their structure. As a result, they may exhibit higher emissivity. In general, emissivity values greater than 0.8 are typical of ordinary nonmetallic materials, including concrete (0.85), glass (0.85-0.95), porcelain (0.92), plastics (0.90-0.97), polypropylene (0.97), polyethylene (0.92), polycarbonate (0.85-0.90), composites (0.85-0.92), Pyrex (0.92), PVC (0.91-0.93), rubber (0.90-0.94), silicone (0.85-0.95), wood (0.88-0.95), or paper (0.91).

By definition, emissivity (ε) is the ratio of the radiant energy emitted by a surface to that emitted by a black body at the same temperature, as described by the Stefan-Boltzmann law. At room temperature (approximately 25° C.), all objects emit thermal radiation primarily in the 3-1000 μm wavelength range and exhibit emissivity values less than 1.0, while an emissivity of 1.0 is theoretically assigned to an ideal black body.

Reference material 13 may also assume various shapes and be of any size. However, to ensure that the thermal image accurately reflects the surface-emitted radiant energy without distortion, a planar geometry with a flat front surface area of at least 50 mm×30 mm is preferred.

In some embodiments, the infrared imaging device 31 is an infrared camera equipped with an IR detector, typically in the form of a focal plane array (FPA) composed of micron-scale detecting elements or “pixels.” The infrared camera employed in the present invention utilizes FPAs that are sensitive to mid- and long-wavelength infrared radiation. While the desired spectral response spans 3-16 μm, the 8-14 μm band is often selected for practical implementation.

The most common types of IR detectors include InSb, InGaAs, HgCdTe, and QWIP FPAs, with selection based on the infrared region to be detected. For example, indium gallium arsenide (InGaAs) operates in the 1.1-1.7 μm range, while indium antimonide (InSb) is sensitive to 1-5 μm. Variants of mercury cadmium telluride (Hg₁₋ₓCdₓTe) can cover detection windows of 1.5-1.8 μm, 2.2-2.4 μm, 3-5 μm, and 8-12 μm. Quantum Well Infrared Photodetectors (QWIPs), typically fabricated from gallium arsenide (GaAs), can be engineered to detect infrared radiation in the 3-20 μm range.

The resolution of the IR camera may range from 160×120 or 320×240 pixels, up to 1280×1024 pixels in some embodiments. A preferred dynamic range exceeds 14 bits, enabling the generation of at least 16,000 grayscale levels in the resulting image.

The infrared camera converts IR radiation into visible images that illustrate the spatial distribution of temperature differences within the observed scene (field of view). FIG. 2 depicts a thermal image captured by the infrared camera, showing variations in brightness (grayscale) corresponding to the test specimen A 41 and reference material 43, positioned in front of the background scene 44, thereby indicating their respective FIR radiation levels.

In practice, it is important to match the IR detector's spectral response with one of the two atmospheric transmission windows: the mid-wavelength infrared (MWIR) band, typically spanning 3-5 μm, or the long-wavelength infrared (LWIR) band, covering approximately 8-14 μm.

Blackbody radiation has a specific, continuous spectrum of wavelengths that depend only on the body's temperature and is governed by Planck's law. The Planck's law (Planck Distribution) can be expressed in terms of wavelength (A) and temperature (T):

$${B}_{Λ} ( λ , T ) = ( 2 {hc}^{2} / {γ}^{5} ) \{ 1 / [ exp ( hc / λ {k}_{B} T ) - 1 ] \} \qquad ( Equation 1 )$$

Where h=6.626070×10⁻³⁴ J.Hz⁻¹ (Planck constant)
K_{B}=1.380649×10⁻²³ J.K⁻¹ (Boltzmann constant)
λ=wavelength, c=speed of light, and T=Temperature (K)

Additionally, the radiant power of thermal radiation from reference material 13 can be described using the Stefan-Boltzmann law:

$$P = A σ ε {T}^{4} \qquad ( Equation 2 )$$

where P=radiant power, A=surface area, ε=emissivity
T=temperature in K, and σ=Stefan-Boltzmann constant.

The theoretical Planck distribution for blackbody (thermal) radiation at a given temperature T can be calculated using the formula above, which is represented by the grayscale values in the image of reference material 43 from the reference material 13.

Meanwhile, the nonthermal FIR-photon radiation from test specimen A 11 passes through reference material 13 substantially unaltered, allowing its thermal image 41 to be superimposed over image of reference material 43 of the reference material. The grayscale values in image of test specimen A 41 represent the total FIR radiation of test specimen (thermal plus nonthermal) and can be quantified by computing the correlation between the grayscale values of images 43 and 41.

To quantify this, assume the grayscale values for images 43 and 41 are V_{RM} (reference material) and V_{TS} (test specimen), respectively. The power of thermal radiation for the reference material 13, P_{RM}, at temperature T is given by Equation 1. Based on this, the power of the total FIR radiation of test specimen, P_{TS}, can then be calculated using the following relation:

$${P}_{RM} / {P}_{T S} = {V}_{RM} / {V}_{TS} ; or {P}_{T S} = ( {V}_{T S} / {V}_{RM} ) {P}_{RM} \qquad ( Equation 3 )$$

The total radiant power measured for the test specimen, P_{TS}, comprises both the nonthermal FIR-photon radiation emitted by the test specimen and the thermal radiation transmitted from the reference material 13. Therefore, the nonthermal FIR-photon radiation can be expressed as: Pₙₒₙₜₕₑᵣₘₐₗ=P_{TS}−P_{RM}.

Based on Equation 2, the effective emissivity of the test specimen, εₜₛ, is given by:

$${P}_{RM} / {P}_{T S} = {ε}_{r m} / {ε}_{t s} ; or {ε}_{t s} = ( {P}_{T S} / {P}_{RM} ) {ε}_{r m} \qquad ( Equation 4 )$$

where εᵣₘ is the known emissivity of the reference material 13.

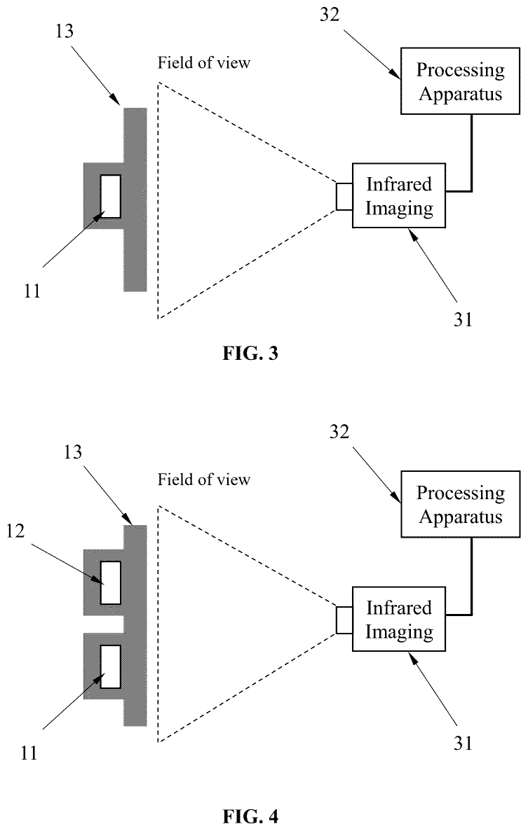

FIG. 3 shows a schematic diagram illustrating other embodiment of the present invention, wherein test specimen A 11 is positioned within reference material 13. The reference material 13 is molded to accommodate test specimen A 11 so that different test specimens of similar shapes can be interchangeably inserted into reference material 13 to ensure accuracy and repeatability of the test results.

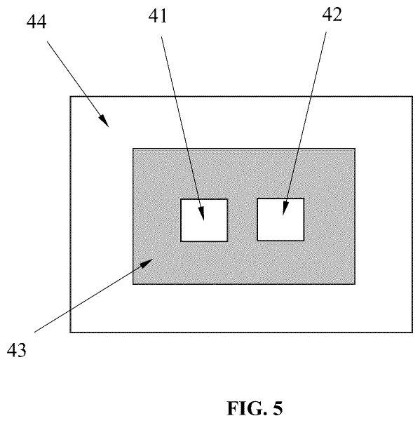

FIG. 4 is a schematic diagram illustrating another embodiment, wherein multiple test specimens are positioned within the reference material to facilitate comparative analysis. For example, two test specimens, 11 and 12, are inserted into reference material 13 for comparison. This approach serves as a crucial tool for the design and development of new composites by analyzing the nonthermal FIR-photon emissions of both specimens.

Accordingly, FIG. 5 displays a thermal image captured by the infrared camera, showing variations in brightness (grayscale) corresponding to the test specimens 41 and 42, and reference material 43, positioned in front of the background scene 44, thereby indicating their respective FIR radiation levels.

The images of test specimens 11 and 12 are presented side by side, enabling direct comparison based on their grayscale values. A higher grayscale value indicates a greater level of nonthermal FIR-photon emission relative to the other specimen. Furthermore, in practical applications, analyzing multiple specimens with varying parameters can effectively reveal trends in the fabrication process. Here, “multiple specimens” refers to groups of 2, 3, 4, 5, or more test samples.

In one embodiment, infrared imaging device 31 features a VOx (Vanadium Oxide) UFPA operating in the 8-14 μm spectral band, with a pixel resolution of 640×512 and a 14-bit dynamic range.

The processing apparatus 32 is configured to execute a set of instructions for performing operations, including acquiring thermal images 41, 42, 43, and/or 44 using infrared imaging device 31, calculating the grayscale values of the thermal images 41, 42, 43, and/or 44, and processing data to generate a display for both the test specimen(s) 11 and/or 12 and reference material 13, as disclosed above. By design, the system can easily determine whether a test specimen is a nonthermal FIR-photon emitting material (i.e., persistent FIR-luminescent material) and quickly assess its radiant power and effective emissivity.

In another experiment, a MWIR detector, Mercury Cadmium Telluride (HgCdTe), was used to measure FIR-photon radiation within the 3-5 μm wavelength range from the same test specimen(s) 11 and/or 12 and reference material 13.

Based on the results from the experiments conducted in the MWIR (3-5 μm) and LWIR (8-14 μm) spectral ranges, these findings support the conclusion that the test specimen emits nonthermal FIR-photons within the 3-16 μm range. This emission, detectable using the method and system of the present invention, would otherwise remain undetected using conventional techniques.

### Conclusion, Ramifications, and Scope

According to the present invention, a method and system for detecting and measuring nonthermal far-infrared photon radiation from the surface of a test specimen comprise at least a test specimen, a reference material, an infrared imaging device, and a processing apparatus configured to execute a set of instructions for performing operations, including capturing thermal images of the test specimen and reference material, and computing their grayscale values to produce results that can determine the radiant power and emissivity of the test specimen.

The present invention provides a crucial tool for the detection and measurement of nonthermal FIR-photon radiation in the 3-16 μm wavelength range, which would otherwise be undetectable and imperceptible.

The invention has been described above. Numerous modifications and variations of the present invention are possible in light of the above teachings. These variations should not be considered a departure from the spirit and scope of the invention, and all modifications that would be obvious to one skilled in the art are intended to be included within the scope of the following claims.

1. A method for measuring nonthermal far-infrared radiation emitting from the surface of a specimen comprising:
a) providing a test specimen;
b) providing a reference material;
c) providing an infrared imaging device to capture thermal images of the test specimen and reference material
d) positioning the reference material and test specimen such that the test specimen and reference material generate thermal image data which is intercepted by the infrared imaging device;
e) providing a processing apparatus to perform operations, including acquiring, computing, and displaying said thermal image data;
wherein steps a) through e) produce results that can be used to quantify a radiant power and an emissivity of the nonthermal far-infrared radiation emitted by the test specimen.

2. The method of claim 1, wherein:
the reference material is a nonmetal, such as polymers, ceramics, glass, composites, concrete, wood, or paper.

3. The method of claim 1, wherein:
the reference material consists of polymers, such as plastics, polyethylene, polycarbonate, rubber, or silicone.

4. The method of claim 1, wherein:
the reference material has an emissivity greater than approximately 0.8.

5. The method of claim 1, wherein:
the reference material is placed between the test specimen and infrared imaging device.

6. The method of claim 1, wherein:
the test specimen is inserted into the reference material.

7. The method of claim 1, wherein:
the infrared imaging device is an infrared camera having a spectral band covering at least a part of 3-16 μm wavelength spectrum.

8. The method of claim 7, wherein:
the infrared camera has a spectral band in about 8 μm to about 14 μm wavelength range.

9. The method of claim 7, wherein:
the infrared camera has a spectral band in about 3 μm to about 5 μm wavelength range.

10. A system for measuring nonthermal far-infrared radiation emitting from the surface of a test specimen comprising:
a) a reference material,
b) an infrared imaging device, and
d) a processing apparatus configured to execute a set of instructions for performing operations, including capturing thermal images of the test specimen and reference material, computing the gray values for both, and producing results that can be used to quantify the radiant power and emissivity of the nonthermal far-infrared radiation emitted by the test specimen.

11. The system of claim 10, wherein:
the infrared imaging device is an infrared camera having a spectral band covering at least a part of about 3 μm to about 16 μm wavelength range.

12. The system of claim 11, wherein:
the infrared camera has a spectral band in about 8 μm to about 14 μm wavelength range.

13. The system of claim 12, wherein:
the infrared camera has a VOx (Vanadium Oxide) UFPA detector in LWIR (8-14 μm).

14. The system of claim 11, wherein:
the infrared camera having a spectral band in about 3 μm to about 5 μm wavelength range.

15. The system of claim 14, wherein:
the infrared camera has a mercury cadmium telluride (HgCdTe) FPA detector in MWIR (3-5 μm).

16. The system of claim 10, wherein:
the reference material is a nonmetal, such as polymers, ceramics, glass, composites, concrete, wood, or paper.

17. The system of claim 10, wherein:
the reference material consists of polymers, such as plastics, polyethylene, polycarbonate, rubber, or silicone.

18. The system of claim 10, wherein:
the reference material has an emissivity greater than approximately 0.8.

19. The system of claim 10, wherein:
the reference material is positioned between the test specimen and infrared imaging device.

20. The system of claim 10, wherein
the test specimen is inserted into the reference material.
